(19) United States
(12) Patent Application Publication
(10) Pub. No.: US 2023/0335237 A1
(43) Pub. Date: Oct. 19, 2023

(54) MEDICAL INFORMATION QUERY AND INPUT SYSTEM, MEDICAL INFORMATION QUERY AND INPUT METHOD, AND PROGRAM FOR PERFORMING SAME

(71) Applicant: HD JUNCTION, INC., Seoul (KR)

(72) Inventors: Jee Hyun SHIN, Seoul (KR); Jae Hyung HAN, Seoul (KR)

(21) Appl. No.: 17/796,232

(22) PCT Filed: Jan. 25, 2021
(86) PCT No.: PCT/KR2021/000954

§ 371 Date: Jul. 28, 2022

(30) Foreign Application Priority Data
Jan. 30, 2020 (KR) 10-2020-0010743
Jan. 30, 2020 (KR) 10-2020-0124253
Sep. 24, 2020 (KR) 10-2020-0124254

(51) Int. Cl. G16H 10/60; G16H 15/00

(52) U.S. Cl. CPC G16H 10/60; G16H 15/00

(57) ABSTRACT

The present invention relates to a medical information query and input method, and a program and a system for performing same, and provided are a medical information query and input method, and a program and a system for performing same, the medical information query and input method comprising the steps of: retrieving, from a database, a patient’s medical information prior to a reference date; generating, from the patient’s medical information, each object by date according to a set field item; and serially displaying, on an interface, each of the objects in a separate section form by date in a corresponding field.

### BACKGROUND OF THE DISCLOSURE

### Field of the Disclosure

The present invention relates to a medical information query and input method and a program for performing the same, and more specifically, to a medical information query and input system, a query and input method, and a program for performing the same for improving readability and user convenience.

### Related Art

While medical information on patients was conventionally managed by being written on paper, computerization has been achieved in the form of an electronic medical record at present. A system for querying, recording and using such an electronic medical record is constructed in hospitals, and a user accesses the electronic medical record through a user terminal such as a computer to query and record the electronic medical record.

However, an interface of a conventional system for querying and inputting electronic medical records is configured in the form of a library, and thus there is a problem that a procedure for accessing a desired record is cumbersome. In addition, since medical information is displayed through different windows according to dates or medical information types, it is difficult for a user to check a time-series change in a record and give medical treatment. There is a limit to access and use such a conventional system interface by an unprofessional user such as a patient due to a complicated structure thereof and there is a problem of deterioration of adaptability to user terminals diversified into tablet PCs, mobile phones, and the like.

### SUMMARY

To solve the aforementioned problems, the present invention provides a medical information query and input system, a query and input method, and a program for performing the same which can easily query desired medical information of a user and check a medical history and a state of a patient in a time series.

The present invention provides a medical information query and input method including: retrieving, from a database, medical information on a patient before a reference date; displaying the medical information on the patient in the form of a plurality of sections divided by dates and by fields on an interface of a terminal; and inputting information corresponding to the reference date or information after the reference date using sections prior to the reference date displayed on the interface.

Here, the plurality of sections are arranged by dates and displayed on the interface, an area to which information corresponding to the reference date or information after the reference date is input is separated from an area in which information before the reference date is displayed, and while sections before the reference date are moved by a scroll operation of a user, the area to which information corresponding to the reference date or information after the reference date is input is maintained in a fixed state.

In addition, the area to which information corresponding to the reference date or information after the reference date is input is arranged on the right side of the area in which information before the reference date is displayed on the interface.

In the input step, a section before the reference date displayed on the interface is selected and moved to a position corresponding to the reference date or a position after the reference date in a drag-and-drop manner, and information corresponding to the reference date or information after the reference date is input.

Here, the section before the reference date includes a plurality of pieces of unit information, and each of the unit information is displayed as a recognizable separate object, and thus the user can select an entire section in the input step or select some unit information from the section.

In the input step, when the user selects the entire section, moves the section in a dragging manner and inputs the section, all unit information included in the section is input to information corresponding to the reference date or information after the reference date, and when the user selects some unit information, moves the same in a dragging manner and inputs the same, the selected some unit information is input to information corresponding to the reference date or information after the reference date.

The method further includes automatically arranging unit information corresponding to the reference date or unit information after the reference date input through the input step according to a preset arrangement criterion.

The inputting of information corresponding to the reference date or information after the reference date further includes calling an input window for a corresponding field and inputting information through the input window.

Meanwhile, the information corresponding to the reference date or the information after the reference date input in the input step includes information on a prescription field, and when the input prescription field includes an examination item, a separate section is generated in an examination field corresponding to the reference date or an examination field after the reference date. The generated section of the examination field includes information on whether an examination has been completed.

The present invention provides a medical information query and input program including: an interface for displaying medical information on a patient in a form of separate sections according to date information and field information and allowing a user to input medical information on a patient through the interface; and a processor for processing medical information on a patient retrieved from a database such that the medical information can be displayed on the interface, wherein, when the user inputs information corresponding to a reference date or information after reference date using a section before the reference date on the interface, the processor generates a section corresponding to the reference date or a section after the reference date based on the input information and displays the section on the interface.

Here, the interface includes an area in which information before the reference date is displayed and an area for inputting information corresponding to the reference date or information after the reference date, and the two areas are separated from each other such that the area to which information corresponding to the reference date or information after the reference date is input is maintained in a fixed state while a section in the area in which information before the reference date is displayed is moved by user operation.

The processor may include: an input processing unit for processing information input by a user through the interface; and an object generation unit for generating, from medical information on a patient retrieved from the database or input through the interface, each object corresponding to date information and field information such that the object is displayed in the section on the interface, wherein, when the user selects a section before the reference date on the interface and moves the same to the area for inputting information corresponding to the reference date or information after the reference date in a drag-and-drop manner, the input processing unit processes the information corresponding to the section as being input, and the object generation unit generates an object for the input information such that the input information can be displayed on the interface.

In addition, a section before the reference date includes a plurality of pieces of unit information, and each of the unit information is displayed as a recognizable separate object such that the user can select the entire section and move the same to input information corresponding to the reference date or information after the reference date or can select some unit information from the section and move the same to input information corresponding to the reference date or information after the reference date.

Meanwhile, the input processing unit processes the content of the entire selected section as being input when the user selects and inputs the entire section, and processes the content corresponding to selected some unit information as being input when the user selects and inputs some unit information.

In addition, the object generation unit generates objects such that input unit information corresponding to the reference date or unit information after the reference date can be automatically arranged and displayed according to a preset alignment criterion.

The present invention provides a medical information query and input program including: an interface displaying medical information on a patient in the form of separate sections according to date information and field information and including a mode selector for selecting a mode for displaying the medical information on the patient; and a processor for processing medical information on a patient retrieved from the database such that the medical information can be displayed on the interface based on a mode selected by the mode selector.

In addition, the mode selector is configured to allow a user to select one of a first mode and a second mode, wherein basic medical information on a patient is displayed on the interface by dates and by fields in the first mode, and relevant field information among the basic medical information on the patient is extracted and displayed based on a representative value selected by the user in the second mode.

The mode selector is configured to allow the user to further select a (1-1)-th mode, wherein the basic medical information on the patient is displayed by dates on the interface and is aligned upward such that a space due to absent field information among medical information by dates is minimized in the (1-1)-th mode.

The mode selector provides a representative value selection window through which the user can select a representative value when the second mode is selected. In addition, the mode selector provides a relevant field selection window through which relevant field information corresponding to the representative value selected by the user can be modified when the second mode is selected.

The present invention provides a medical record query and input method including: retrieving medical information on a patient from a database; classifying the medical information on the patient by dates and by fields and displaying the medical information in the form of separate sections on the interface in time series; selecting a representative value by a user through the interface; and extracting a section corresponding to a field associated with the representative value and displaying the extracted section on the interface.

Here, the representative value selected by the user may be one of a medical subject, a diagnosis name, an examination name, a treatment name, and a medicine name.

Relevant field information for each representative value is preset, and the displaying of the extracted section may include extracting and displaying a section for preset relevant field information corresponding to the selected representative value.

In addition, the relevant field information may be modified by the user through the interface, and the displaying of the extracted section may include extracting and displaying a section for the relevant field information modified by the user.

The present invention provides a medical record query and input method including: retrieving medical information on a patient from a database; generating objects to be displayed in the form of sections on an interface of a user terminal based on the retrieved medical information; selecting a representative value by a user through the interface; and displaying an object related to the representative value in the form of a section on the interface.

In addition, the displaying in the form of a section on the interface includes two-dimensionally classifying and displaying a plurality of sections according to date information and field information.

Each of the objects includes date information and field information, and the displaying in the form of a section on the interface may include selectively displaying an object corresponding to a field related to the representative value in the form of a section on the interface.

In addition, the representative value selected by the user may be one of a medical subject, a diagnosis name, an examination name, a treatment name, and a medicine name.

Relevant field information is preset for each representative value, and the displaying of the object related to the representative value in the form of a section on the interface may include selectively displaying a section corresponding to the preset relevant field information.

The relevant field information may be modified by a user through the interface, and the displaying of the object related to the representative value in the form of a section on the interface may include selectively displaying a section corresponding to the modified relevant field information.

The present invention includes the steps of: retrieving medical information on a patient before a reference date from a database; generating a plurality of objects by extracting or processing some of the retrieved medical information such that information by dates and by fields among the retrieved medical information can be displayed on an interface of a terminal according to a preset policy; and displaying the respective objects in the form of separate sections on the interface.

In addition, an object of a preset field is generated such that it includes results of comparing information before a corresponding date with information corresponding to the corresponding date, and a section of the preset field on the interface displays the results of comparing information before a corresponding date with information corresponding to the corresponding date on the interface.

The preset field is one of fields for displaying examination results of the patient, and the section of the preset field may display information on an examination information corresponding to the corresponding date and information regarding increase/decrease compared to an examination result before the corresponding date.

The information on the examination result corresponding to the corresponding date is indicated as numbers, and the information regarding increase/decrease is indicated as a symbol.

In addition, the section of the preset field may further display information on whether the examination result corresponding to the corresponding date is within a normal range.

In addition, the method may further include displaying detailed information of a section selected by a user on the interface through a detailed display window, wherein the amount of information displayed through the detailed display window may be greater than the amount of information displayed in the section.

A section corresponding to a diagnosis information field among the sections may display information on a main diagnosis name and a sub-diagnosis name or the main diagnosis name and a similar symptom.

In addition, the section may display the main diagnosis name and the sub-diagnosis name or the main diagnosis name and the similar symptom in a distinguishable manner using any one of a symbol, a color, and a letter shape.

The present invention provides a medical information query and input program including: an interface displaying medical information on a patient in a form of separate sections according to date information and field information and allowing a user to input medical information on a patient; and a processor for processing medical information on a patient retrieved from a database such that the medical information can be displayed on the interface, wherein the processor includes an object generation unit for generating a plurality of objects corresponding to respective sections by extracting or processing some of the retrieved medical information such that each section can be displayed on the interface according to a preset policy.

In addition, in generation of an object of a preset field, the object generation unit generates the object such that object includes results of comparing information corresponding to a corresponding date with information before the corresponding date, and a section of the preset field displayed on the interface is configured to display the comparison results.

In addition, the preset field is one of fields displaying examination results of the patient, and the section of the preset field displays information on an examination result corresponding to the corresponding date and information regarding increase/decrease compared to examination results before the corresponding date.

In addition, the information on the examination result corresponding to the corresponding date is indicated as numbers and the comparison information is indicated as a symbol.

The section of the preset field further displays information on whether the examination result corresponding to the corresponding date is within a normal range.

In addition, the interface is configured to selectively display a detailed display window for displaying detailed information of a section selected by a user, and the amount of information displayed through the detailed display window may be greater than the amount of information displayed in the section.

A section corresponding to a diagnosis information display field among the sections displays a main diagnosis name and a sub-diagnosis name or the main diagnosis name and a similar symptom, the section displaying the main diagnosis name and the sub-diagnosis name or the main diagnosis name and the similar symptom in a distinguishable manner using any one of a symbol, a color, and a letter shape.

The present invention provides a medical information query and input method including: retrieving, from a database, medical information including examination information on a patient before a reference date; generating objects by dates and by fields from the retrieved medical information on the patient; displaying the objects in the form of separate sections by dates on an interface of a terminal; and displaying examination result information by dates corresponding to an examination field selected by a user as a graph through a separate display window.

The retrieved medical information includes treatment information by dates or prescription information by dates, and the displaying as a graph includes displaying the treatment information by dates or the prescription information by dates on the same time axis as the examination result information by dates.

The examination result includes results with respect to a plurality of detailed examination items, and the displaying as a graph includes displaying a separate graph for each detailed examination item, and when the user selects one of treatment information and prescription information, a graph of a detailed examination item associated with the selected treatment information or prescription information is displayed to be identified discriminately from graphs of other detailed examination items.

The displaying as a graph includes displaying a plurality of graphs corresponding to the detailed inspection items on the same time axis in an overlapping manner.

### Advantageous Effects

According to the present invention, a user can easily ascertain temporal changes in related information and correlation between pieces of field information because medical information of multiple dates can be simultaneously checked through one interface screen in a time series.

Furthermore, a display on an interface has a structure similar to a conventional paper medical chart in the present invention, and thus a user-familiar use environment can be provided and a user can easily identify information for each field through the display.

In addition, since new input can be performed using existing patient medical information according to the present invention, information can be easily input and the present invention can be easily used in various device environments such as a touchscreen.

### BRIEF DESCRIPTION OF THE DRAWINGS

FIG. 1 is a diagram showing a configuration of a medical information query and input system according to an embodiment of the present invention.

FIG. 2 is a block diagram showing major components of a user terminal of FIG. 1.

FIG. 3 is a diagram showing an interface of FIG. 2.

FIG. 4 is a diagram showing a display of details of some sections in FIG. 3.

FIG. 5 is a diagram showing a time-series display of some examination results through an additional window in FIG. 3.

FIG. 6 is a flowchart showing a medical information query and input method performed by the system of FIG. 1.

FIG. 7 is a diagram showing an example of inputting details of a first field to an interface.

FIG. 8 is a diagram showing another example of inputting details of the first field to the interface.

FIG. 9 is a diagram showing an example of inputting details of a second field to the interface.

FIG. 10 is a diagram showing an example of inputting the details of the second field to the interface.

FIG. 11 is a flowchart illustrating the inputting operation described in FIGS. 8 to 10.

FIG. 12 is a diagram showing an interface displaying information related to representative values selected by a user.

FIG. 13 is a flowchart showing a method of selectively displaying the information related to representative values selected by the user.

FIG. 14 is a diagram showing a display mode in which information is aligned upward in a first display mode.

FIG. 15 is a diagram showing a mode in which information is aligned by fields in the first display mode.

FIG. 16 is a diagram showing setting of a filter value in a second display mode.

FIG. 17 is a diagram showing a display mode in which information is aligned upward in the second display mode.

FIG. 18 is a diagram showing a mode in which information is aligned by fields in the second display mode.

FIG. 19 is a diagram showing an example of an interface in which diagnosis and examination information is displayed.

FIG. 20 is a diagram showing an example of an interface in which examination information is displayed through a detailed inquiry window.

FIG. 21 shows a graph displayed for specific examination items.

### DESCRIPTION OF EXEMPLARY EMBODIMENTS

Hereinafter, a medical information query and input system, a query and input method, and a program for performing the same according to embodiments of the present invention will be described in detail with reference to the drawings. In the following description, positional relation of components will be described on the basis of the drawings in principle. In addition, in the drawings, structures of the invention may be simplified or exaggerated as necessary for convenience. Accordingly, the present invention is not limited thereto and various devices may be added, modified, or omitted.

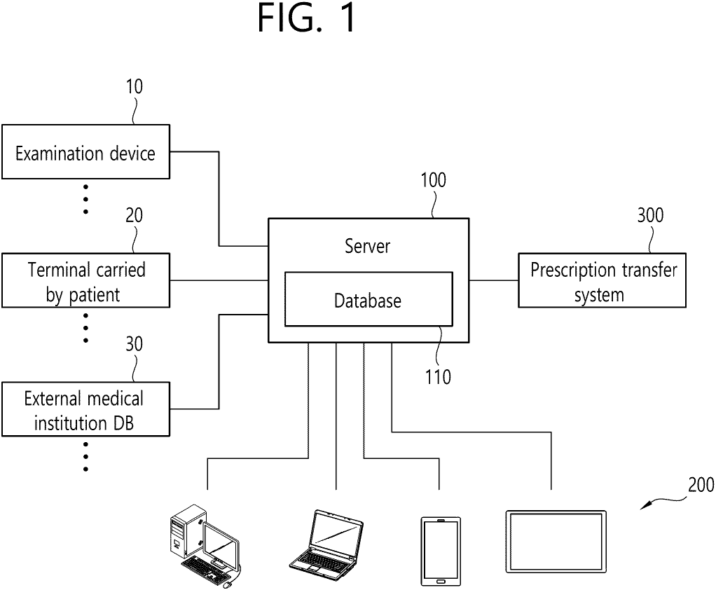

FIG. 1 is a diagram showing a configuration of a medical information query and input system according to an embodiment of the present invention. As illustrated in FIG. 1, a server 100 is configured to be connected to various examination devices 10, various terminals 20 carried by patients, servers 30 of external medical institutions, and a plurality of user terminals 200 using a network to receive information. This server 100 includes a database 110 for storing various types of medical information and stores various types of medical information about patients provided through the network in the database 110. Data stored in the database 110 may include various types of information about personal information and past medical histories of patients. For example, the data may include information personally recorded by medical personnel, such as medical records, operation records, nursing records, and anesthesia records. Alternatively, the data may include result information about various examinations such as image examination and functional examination, and information about details of treatment and details of prescription performed at the time of past medical treatment. Furthermore, the data may include medical information about patients received from external medical institutions and information about various measurement results and request details transmitted through devices carried by patients.

Although FIG. 1 illustrates a structure in which the server 100 is connected to the plurality of user terminals 200, the server may be connected to a single user terminal in a small medical institution such as a private hospital. Furthermore, although FIG. 1 illustrates a configuration in which the server and the user terminals are separate devices, a user terminal may include a database so that it can serve as both a server and a user terminal. Although such a server can be configured as hardware included in a medical institution, the server may be configured using a cloud server on the Internet such that a user can access it anywhere through the Internet.

The user terminals 200 are devices by which users can query medical information about patients and input medical information through an interface. These user terminals 200 can be configured using various electronic devices by which information can be displayed to users and users can input information, such as a desktop computer, a notebook computer, a tablet PC, and a mobile phone. Although the user terminals 200 are user terminals used by medical personnel in the description of the present embodiment, the present invention is not limited thereto and they may be configured as user terminals by which patients can query their medical record information and input information.

Medical information about patients input through the user terminals 200 can be newly stored or upgraded in the database 110. Further, when order information such as medical treatment details, prescription details, and reservation is input through the user terminal 200, the corresponding information can be transmitted to a prescription transfer system 300 through the server 100 such that the corresponding order can be performed.

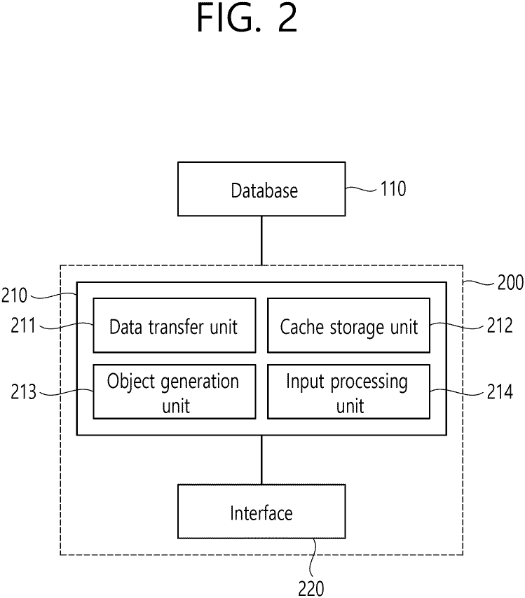

FIG. 2 is a block diagram showing major components of the user terminal of FIG. 1. As illustrated in FIG. 2, the user terminal 200 may include a processor 210 and an interface 220.

The processor 210 is a component that sends a request for medical information about a patient requested through the interface 220 to the database 110 and processes the medical information about the patient provided through the database 110 such that the medical information can be displayed through the interface 220. Here, the database 110 is indicated using the reference sign of the database of the server for convenience but it is not limited thereto and may be a database of another external system or a database included in the user terminal. In addition, the processor 210 is a component that transmits input information input through the interface 220 such that the input information can be stored in the database 110 and generates and transfers order information such that order details among the input information can be transmitted to the prescription transfer system 300.

In addition, the interface 220 is a component that displays medical information to a user through a display of the user terminal 200. Further, the user can input information such as a medical record, medical treatment details, and prescription details through the interface 220. Here, the user can input various types of information through the interface using a keyboard, a mouse, an input pen, or the like, and if the display is configured as a touchscreen, may input information through touch.

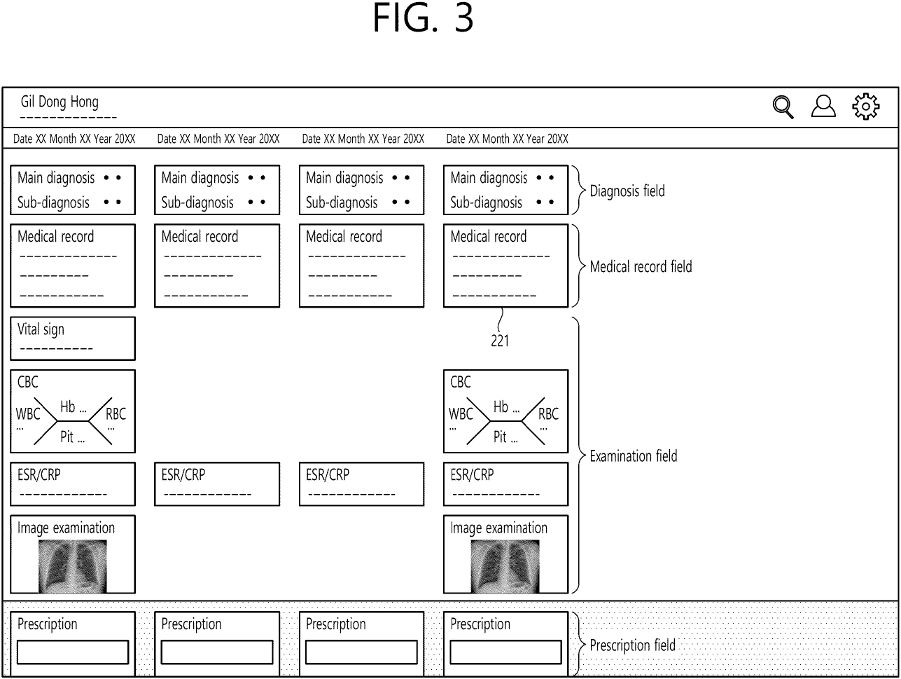

FIG. 3 is a diagram showing the interface of FIG. 2. As illustrated in FIG. 3, patient information such as the name, sex, and age of a patient is displayed and selection icons through which various functions can be selected are disposed at the top of the interface 220. In addition, a space in which various types of medical information of a selected patient are displayed or recorded is formed in the lower part. Such medical information about a patient includes various types of medical history information such as a diagnosis record, an examination record, an operation record, and a hospitalization record. This medical information is displayed in such a manner that events corresponding to a medical history are displayed by dates along a time axis in the horizontal direction, as illustrated in FIG. 3. Accordingly, a user can check the medical history of the patient through one interface screen in a time series.

Specifically, dates on which events corresponding to the medical history of the patient occur are displayed along the time axis. In general, a reference date is displayed at a position a predetermined distance from the left side of the time axis and dates prior to the reference date are displayed on the left of the reference date. Here, the reference date may be a set date and is generally the day when a user queries or inputs medical information. In addition, medical information corresponding to each date is displayed under each date in the horizontal direction. Here, the vertical axis is divided into a plurality of fields according to details and characteristics of medical information. These fields may include a diagnosis field, a medical record field, an examination field, a prescription field, and the like. Accordingly, medical information about patients stored in the database is classified according to characteristics thereof and displayed at positions of corresponding fields by dates corresponding to medical histories on the interface. A user can check details corresponding to each field while scrolling medical information by dates in the vertical direction. However, some fields (the prescription field in the present embodiment) may be configured to selectively rise up from the bottom of the interface in consideration of user convenience, as illustrated in FIG. 3 (here, a field is a superordinate concept including a set of subfields and is used as a term discriminated from a subfield but it means classification according to information characteristics and may be used as a term including a subfield itself).

In an interface of a conventional system, a procedure for accessing information through the interface is cumbersome because the interface is configured in the form of a library and it is difficult for a user to perform synthetic determination based on past medical information about a patient because information is individually managed and displayed. On the other hand, according to the present invention, a user can simultaneously check medical information of multiple dates through one interface screen in a time series. Furthermore, it is easy to ascertain temporal changes in related information because medical information about a patient is classified by field and displayed in a time series. In particular, since pieces of information corresponding to various fields are displayed on one screen, a correlation between pieces of information of fields can be easily ascertained (for example, a correlation between blood sugar test level changes according to a prescribed medicine change). Accordingly, a user can easily ascertain a medical history of a patient and details of prescription through the interface according to the present invention and synthetically determine changes in a state of the patient and effectiveness of previous prescription.

Patient information by dates and by fields is provided in the form of a section on the interface. Here, provision of a section form means that each piece of information is formed as a separate entity and displayed on the interface such that it can be graphically discriminated from other pieces of information by dates and by fields. In addition, a user can view details of information in units of section and move a position of information on the interface in units of section. For example, information corresponding to each date and each field is displayed as a section 221 in a card shape on a background of the interface, as illustrated in FIG. 3. This structure is similar to a conventional paper medical chart and thus provides a user-familiar use environment, and a user can easily identify information corresponding to each field. However, one section 221 is not configured for each field of a corresponding date, and when a plurality of subfields is present for a field, separate sections are configured for the respective subfields. For example, separate sections can be displayed according to examination types such as blood examination and X-ray examination in the examination field, and separate sections can be displayed according to prescription types such as prescription of medicine, prescription of medical treatment, prescription of examination, and prescription of rehabilitation in the prescription field.

In this manner, a default screen of the interface according to the present embodiment is displayed such that sections are aligned by dates and by fields. Accordingly, there is no information corresponding to some fields on a specific date, and thus, a blank part in which a section is not generated may be generated. Therefore, the interface includes a function of aligning and displaying sections upward such that a blank is not generated between sections of the same date. Accordingly, a user can check medical information about a patient while minimizing scrolling of the screen in the vertical direction by selecting the function in query of records by dates.

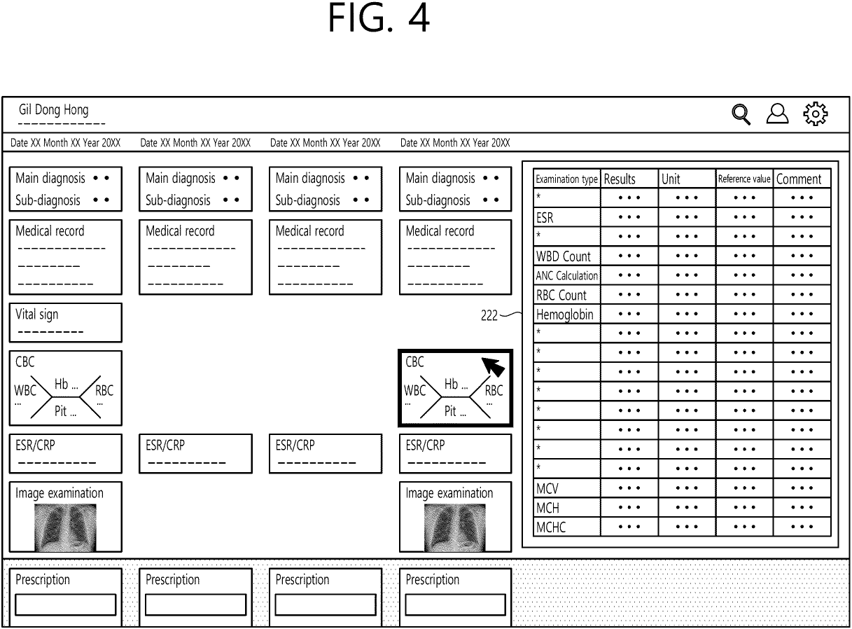

FIG. 4 is a diagram showing a display of details of some sections in FIG. 3. In each section 221 displayed on the interface 220, information of a corresponding date and a corresponding field is processed and displayed. When a user requests query of detailed information of the corresponding section, the detailed information is displayed through an additional detailed query window 222. The request may be performed in such a manner that the user selects the corresponding section, and this selection may be performed through a clicking operation using a mouse, a touch operation through a touchscreen, or the like. The detailed query window 222 may be configured as a separate section generated on one side of medical information about a patient, as illustrated in FIG. 4. Since the detailed query window 222 is overlaid on the default display screen without an additional screen change operation, continuity of the medical information query operation of the user can be maintained.

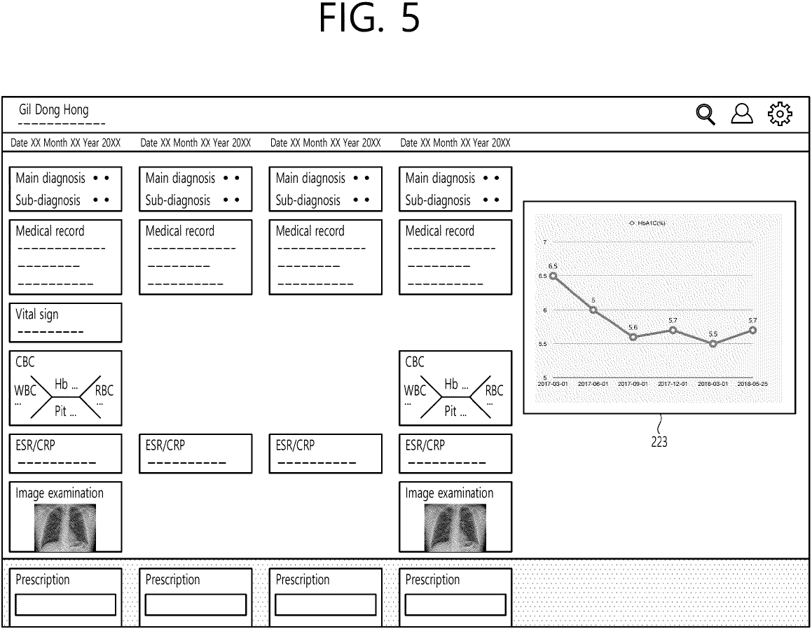

FIG. 5 is a diagram showing a time-series display of some examination results through an additional window in FIG. 3. As described above, the interface of the present embodiment provides medical information by dates and by fields, and thus a user can check changes in each piece of field information in a time series through the default screen of the interface. However, the interface according to the present embodiment may include a function of displaying such changes in more detail through an additional detailed information window 223 for each field. When a user selects a field (e.g., X-ray examination result field) (e.g., clicks a field name), medical information corresponding to the field can be displayed through an additional detailed query window in a time series. In a case where query is performed through the default screen, some sections are displayed as blanks when the corresponding examination was not performed on some dates. However, in a case where information is displayed through an additional detailed query window, corresponding field information can be checked in detail without a blank. Furthermore, more detailed information than content displayed through each section can be displayed through a detailed query window, or changes over time can be displayed as a graph or can be processed into statistics and displayed if corresponding information pertains to numerical values.

A basic display form of the interface according to the present embodiment has been described above in detail with reference to FIGS. 3 and 5. However, the number of dates, time axis information such as displayed date periods, field types, and the like displayed on the interface may be changed according to user settings.

Referring back to FIG. 2, the configuration of the processor is described in more detail. As illustrated in FIG. 2, the processor 210 may include a data transfer unit 211, a cache storage unit 212, an object generation unit 213, and an input processing unit 214.

The data transfer unit 211 is a component that is connected to various databases (including databases in user terminals, servers, additional examination devices, webs, etc.) in a wired/wireless manner and transmits and receives necessary information. Accordingly, when a user requests medical information about a specific patient through the interface 220, the data transfer unit 211 of the processor 210 retrieves the information from the database 110. Then, the data transfer unit 211 transmits the medical information input through the interface 220 such that the medical information can be stored in the database 110. Furthermore, when the user inputs a prescription order through the interface 220, the data transfer unit 211 can execute a function of transmitting the order to the prescription transfer system 300.

The cache storage unit 212 is a component that temporarily stores medical information about a patient transmitted from the database 110. Accordingly, the processor 210 can extract and process information using medical information stored in the cache storage unit according to settings through the interface or a request of a user. In addition, when a user inputs medical information, the processor 210 can reflect the input medical information in medical information stored in the cache storage unit and transmit the medical information stored in the cache storage unit 212 to the database 110 such that medical information in the database can be updated.

The object generation unit 213 is a component that generates an object to be displayed in each section 221 of the interface 220 among medical information about a patient transmitted from the database 110. Some of medical information about a patient transmitted from the database 110 are displayed in a section of the interface and various display formats may be provided according to fields. Accordingly, the processor 210 can extract information by displayed dates and information about fields from medical information stored in the cache storage unit 212 and the object generation unit 213 can generate an object to be displayed in a section for each date and each field in a display format defined for each field.

For example, results with respect to blood examination in the examination field may include various types of information as displayed in a detailed query window, as illustrated in FIG. 4. However, a section corresponding to a blood examination item is defined such that only four major items among the blood examination results are displayed in a designated pattern. Accordingly, the object generation unit 213 generates an object such that only corresponding information can be extracted from blood examination result information and displayed in a designated pattern.

An object generated by the object generation unit 213 can be realized in various forms according to the field corresponding thereto. As illustrated in FIG. 4, objects can be generated such that they are differently displayed in chart forms defined according to examination types such as blood examination and basal metabolism profile examination. In addition, a result such as an X-ray imaging result among examination results may be generated such that it is displayed as an image file on a section and a result such as an ultrasonic examination result may be generated such that it is displayed as a moving image reproduced on a section. Further, the diagnosis field and the medical record field may be configured to be displayed in a text form. In this manner, the object generation unit can generate an object for each field and each section scale which will be described later in various forms.

Meanwhile, the input processing unit 214 is a component that processes details input by a user through the interface. The user can perform an input operation such as inputting a diagnosis, a medical record, or prescription corresponding to a reference date through the interface. Details input by the user through the interface in this manner are recognized by the input processing unit, stored in the cache storage unit 212 and processed such that they can be updated to the database 110 or processed to be transmitted to the prescription transfer system 300. Then, the processed information can be transmitted to the database or external components including the prescription transfer system through the data transfer unit 211.

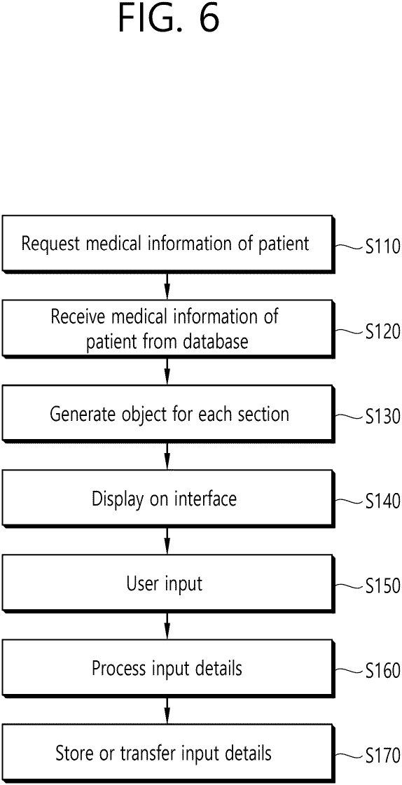

Operations performed by the processor 210 in the medical information query and input system have been described above focusing on major components. FIG. 6 is a flowchart showing a medical information query and input method performed by the system of FIG. 1. Hereinafter, each step will be described in detail with reference to FIG. 6.

First, a step in which a user requests medical information about a patient through the interface is performed (S110). This step may be performed in such a manner that the user inputs the name or ID of the patient through the screen of the interface 220.

The processor 210 retrieves the requested medical information about the patient from the database 110 (S120). As described above, the database 110 may be a database included in a user terminal, a database included in an additional server device, or a cloud server, or a database included in an additional examination device, another medical institution or a device carried by a patient. This step may be performed in such a manner that the medical information is received through the data transfer unit 211 of the processor, and the received medical information may be stored in the cache storage unit 212.

The processor 210 performs a step of generating an object that can be displayed in each section on the interface using the medical information about the patient transmitted from the database (S130). In this step, the processor extracts target information among the medical information about the patient on the basis of set values (e.g., displayed date periods, selected field information, etc.). Here, details included in the target information may be classified by dates and fields (by subfields when there are subfields) in units corresponding to respective sections. The object generation unit 213 generates each object by processing information corresponding to each classified section in a defined form according to the field or subfield corresponding thereto. Here, the defined format may be different according to field types and may be different according to scales set by the user.

Objects generated by the processor 210 are displayed through respective sections of the interface 220 (S140). As described above, details displayed in each section can be generated in various forms such as a text form, a chart form, an image form, and an image reproduction form according to fields or subfields. Accordingly, the user can simultaneously check pieces of information by dates through the interface in a time series and check information by classifying the information by fields according to characteristics thereof.

Meanwhile, the user performs a step of inputting necessary details through the interface 220 while querying the medical information about the patient through the interface 220 (S150). The input details may be a medical record, prescription, or details with respect to a medical reservation or an examination reservation. A specific input method will be additionally described below.

When the user inputs the aforementioned details, the processor 210 processes the input information according to an input field and details (S160). For example, when the user inputs information such as a medical record, the input processing unit 214 processes the information such that this information can be added to existing medical information about the patient stored in the cache storage unit 212 and stored therein. Alternatively, when the user inputs a prescription order or reservation information, the processor 210 generates a command for transferring the corresponding information to the prescription transfer system 300. In addition, the processor 210 can process information input by the user through a method defined according to characteristics of the information.

When processing of the input information is performed in this manner, the processor 210 performs a step of storing the input details or transferring the input details to another component (S170). This step can be performed through the data transfer unit 211 of the processor 210. In this step, content of the cache storage unit storing the medical information about the patient in which the input details have been reflected is transmitted to the database in which the previous medical information about the patient is stored such that the medical information about the patient can be updated. Alternatively, the input details can be transmitted to the prescription transfer system 300 or a device corresponding to the input details.

### Input of Medical Information

Information input by a user through the interface may be largely classified into two. The first one is a field (hereinafter referred to as a first field) through which determination, an opinion and a medical examination by interview of a user, and the like are input, such as a diagnosis field, a medical record field, and a referral field. This is generally input using text and input as medical information corresponding to a reference date in many cases. The other one is a field (hereinafter referred to as a second field) through which a user makes a reservation of examination or rehabilitation on a reference date or after the reference date or inputs a prescription order such as medicine, medical treatment, examination, or rehabilitation, such as an examination field and a prescription field. Hereinafter, methods of inputting details by a user according to characteristics of these fields will be described in detail.

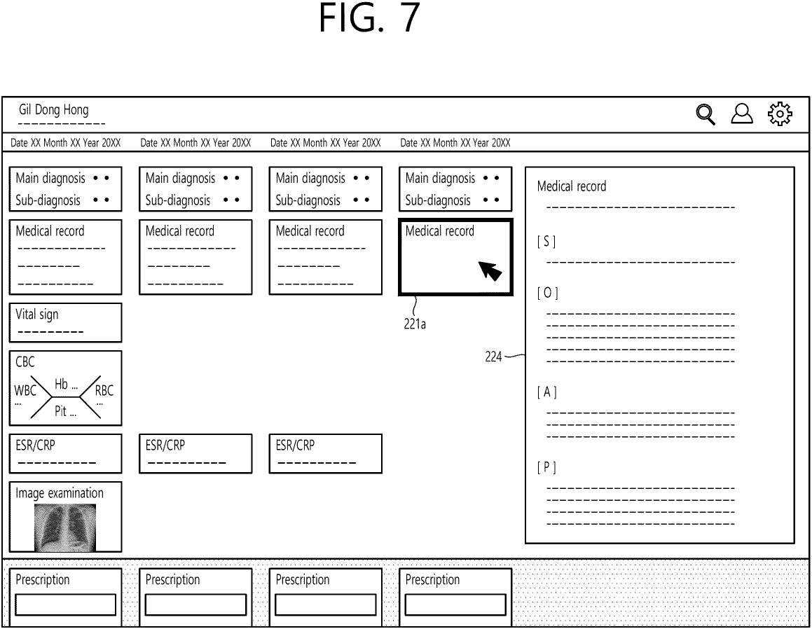

FIG. 7 is a diagram showing an example of inputting details of the first field to the interface (first input method). When medical information about a patient is queried, information prior to a reference date is displayed on the interface in a section format but information corresponding to the reference date and dates after the reference date exists as a blank region unless reserved information is present. Here, if a user selects a blank position of a corresponding field (medical record field) in a reference date column in order to input a medical record corresponding to the reference date during a medical treatment, a vacant section 221a is generated at the corresponding position. If the generated vacant section 221a is selected, an additional input window 224 is displayed on one side of the interface like the aforementioned detailed query window. Accordingly, when the user inputs details of the medical record through the input window 224 and closes the input window 224, an object corresponding to the medical record field of the reference date is generated through the processor and displayed in the corresponding section. The step of generating a vacant section when a blank position is selected may be omitted according to an embodiment. Further, when a blank position is selected, the input window may be immediately displayed when an additional mark (e.g., “+”) indicating addition of a section is displayed and an object reflecting the corresponding details may be generated upon completion of input and a section may be generated at the corresponding field position.

Meanwhile, in a case where a patient frequently visits a medical institution for a specific period of time because of the same lesion, symptoms of the patient may be similar and doctors may have similar opinions in many cases. Accordingly, the interface includes a function of using information input on a previous date instead of newly inputting all details when the first field is input.

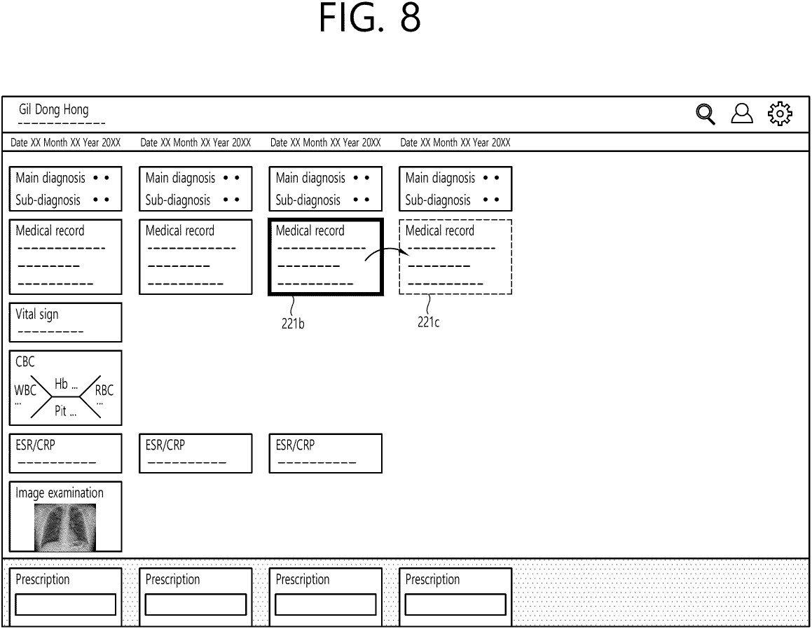

FIG. 8 is a diagram showing another example of inputting the details of the first field to the interface (second input method). When a user intends to input medical information corresponding to a reference date using medical information before the reference date, the user can select a section prior to the reference date which will be used and move the selected section to a corresponding field position of the reference date. This operation may be performed through a conventional drag-and-drop method. A section 221c having the same details as those of an existing section 221b is generated in the same field of the reference date through the aforementioned operation. This corresponds to a state in which the same details as the existing section have been input to the corresponding field of the reference date (this can be used in the case of the same diagnosis). In addition, when the user selects the section 221c generated on the reference date, the input window 224 is displayed on one side, as illustrated in FIG. 7. Here, the input window 224 is in a state in which the same medical information as that corresponding to the existing section 221b has been input, and the user can correct (delete, substitute, or add) the existing details through the input window 224. When the input window 224 is closed, an object corresponding to the corresponding field of the reference date can be regenerated and displayed in the corresponding section 221c.

As described above, according to the present embodiment, user convenience can be improved by performing an input operation using existing medical information and the input operation can be applied to various terminal devices using a method of dragging and dropping a section. Here, movement of an existing section may be limited within the same field in order to prevent improper input.

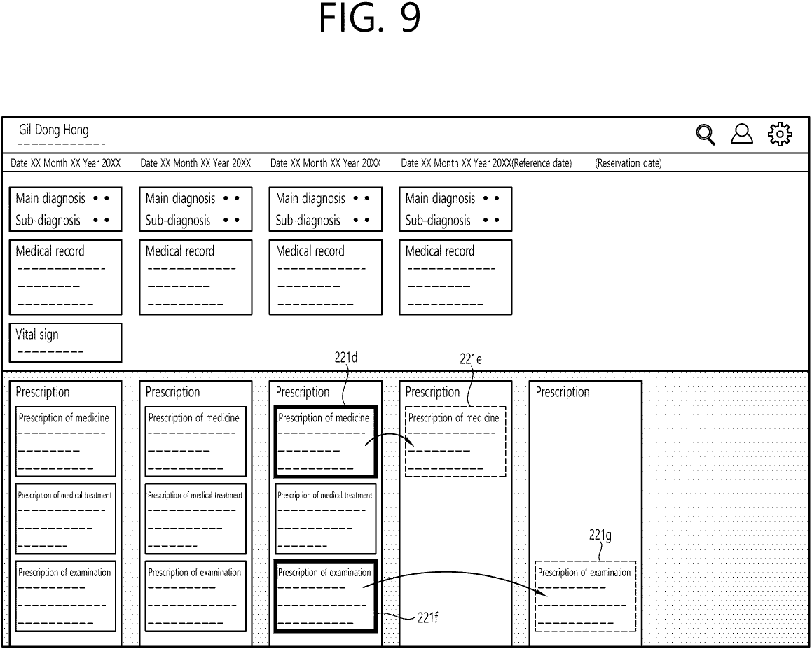

FIG. 9 is a diagram showing an example of inputting details of the second field to the interface. As described above, the second field such as the examination field or the prescription field requires a function of inputting various orders and reservation information corresponding to a reference date or after the reference date. In particular, since details of various prescription orders are highly likely to be similar to prescription performed prior to the reference date, input can be performed using information prior to the reference date as in the aforementioned second input method.

Specifically, FIG. 9 shows prescription field windows and illustrates a state in which windows hidden at the bottom of the interface are selected by a user to rise up. The prescription field may include sub-prescriptions such as prescription of medicine, prescription of medical treatment and prescription of examination. The user can input prescription orders to prescription field windows using the aforementioned second input method.

For example, when the user intends to input a medicine prescription order, the user selects a past section 221d of the medicine prescription field in which a similar prescription is performed and moves the section 221d to a reference date position. Accordingly, a prescription order can be input in such a manner that a section 221e generated at the reference date position is selected and details are corrected. Furthermore, in the case of a medical treatment prescription, a section corresponding to the same medical treatment prescription in the past can be moved to the reference date position and an examination prescription order can be input.

Furthermore, in the case of some subfields that require a reservation function (e.g., prescription of examination and prescription of rehabilitation), a section may be moved to a position after a reference date instead of being moving to the reference date and reservation input may be performed. For example, when examination reservation is required, a past section 221f in which the same examination was prescribed can be moved to a position after the reference date (to the right side of the reference date column) in a prescription field window and the corresponding examination can be reserved. Here, a new section 221g is generated at a position of the corresponding subfield after the reference date and may include even a function of selecting a reservation date if required. In this case, a reservation date can be displayed on the time axis at a position at which a section corresponding to a reservation is generated.

As described above, in the case of the second field, the position of an existing section can be moved, a prescription order can be input and details of reservation can be input through the second input method. Although FIG. 9 mainly illustrates a process of inputting through a prescription field window, a prescription order, and a reservation may be input in the same manner in the examination field. For example, when a user intends to input an examination prescription or an examination reservation in a state in which prescription field windows have not risen up, it is possible to move a section corresponding to the corresponding examination among sections displaying past examination results in the examination field to the reference date or a position after the reference date and input the examination prescription or the examination reservation.

As described above, although movement of a section may be limited within the same field or the same subfield in the second input method, it may be exceptional in examination related fields. Specifically, in the examination field and an examination prescription field of the prescription field, if a user prescribes or reserves a specific examination using one of the fields, a corresponding section can also be generated in the other such that details of prescription or reservation can be checked. Accordingly, the user can prescribe or reserve additional examination without a prescription field window rising up while checking previous examination results through the examination field. In addition, a section generated according to examination prescription or reservation can be displayed to be distinguished from sections displaying previous examination results because examination result information corresponding thereto does not exist yet. For example, while a section displaying results of ultrasonic examination performed before the reference date is displayed such that a moving image is reproduced, a section generated according to examination prescription or examination reservation can be displayed such that an object indicating a vacant section or absence of examination information is displayed. Accordingly, the user can also check whether examination reserved on the reference date at the time of past medical treatment has been performed through a section displayed in the examination field.

Input of details of prescription or reservation to the second field using the second input method has been described above. However, the second field input method is not limited thereto and may also include an input function using the first input method of generating a vacant section at a position corresponding to the reference date or after the reference date and directly inputting information.

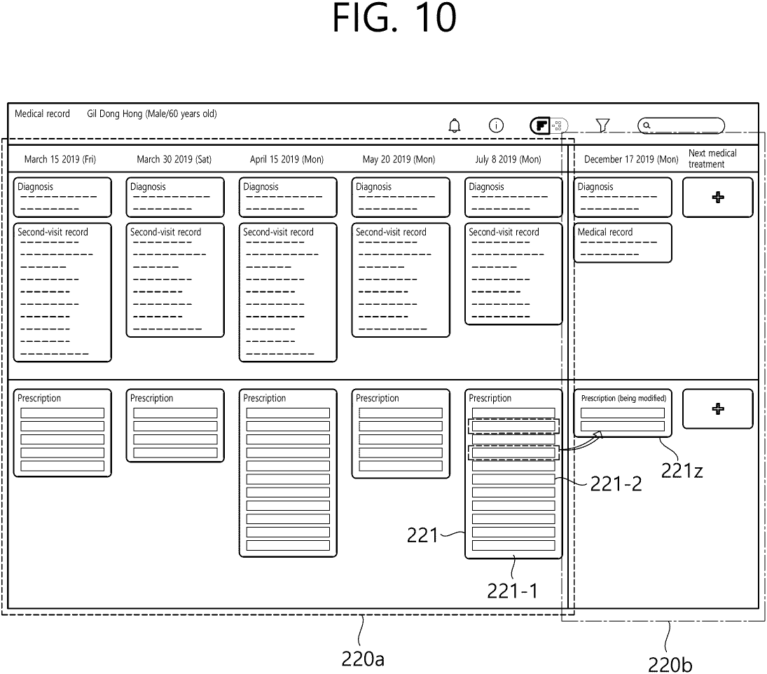

FIG. 10 is a diagram showing an example of inputting the details of the second field to the interface. In FIG. 10, medical information for each date is displayed in time series on a single time axis as in the examples shown in FIGS. 8 and 9, and information by fields among the medical information for each date is displayed as separate sections. Here, although an area in which information before the reference date is displayed and an area in which information corresponding to the reference date is displayed are not separated in FIGS. 8 and 9, an area 220a in which information before the reference date is displayed and an area 220b to which information corresponding to the reference date or after the reference date is input are formed as separate objects in FIG. 10. As shown in FIG. 10, the area in which information before the reference date is displayed is disposed on the left side of the interface, and a plurality of sections corresponding to the information before the reference date are displayed therein. In addition, the area to which information corresponding to the reference date or after the reference date is input is disposed on the right side of the interface separately from the area in which information before the reference date is displayed, and a plurality of sections corresponding to the input information are displayed therein. At this time, even while the area in which the information before the reference date is displayed is moved up, down, left and right by a scrolling operation of the user (for example, when the user intends to inquire information before the date displayed on the interface), the area in which information corresponding to the reference date or after the reference date can be maintained in a fixed state. In addition, when the user intends to call a separate input window and input information corresponding to the reference date or after the reference date (the first input method), the area in which information corresponding to the reference date or after the reference date is displayed can be moved to the left in order to secure a space (for example, the right side of the area in which information corresponding to the reference date or after the reference date) in which the separate input window is displayed. In this case, the area in which information before the reference date is displayed is maintained in a fixed state, and the area in which information corresponding to the reference date or after the reference date can be moved to the left while overlapping with the area in which information before the reference date is displayed. In this way, the area in which information before the reference date is displayed and the area where information corresponding to the reference date or after the reference date are configured as separate areas and can move independently on the interface.

In the embodiment of FIG. 10, it is also possible to input information corresponding to the reference date or information after the reference date through the above-described first input method and second input method. However, although the method of selecting an entire section and moving it in a drag-and-drop manner when information is input using a section before the reference date displayed on the interface is described in FIG. 9, it is also possible to input only selected some information among information included in one section.

Specifically, one section may include a plurality of pieces of unit information. For example, the prescription field may include various types of information such as drug prescription, medical treatment prescription, and examination prescription, and one section displayed in the prescription field of the interface may include various prescription details such as a first examination, a second examination, first medicine, and second medicine. Various types of unit information displayed in one section (herein, unit information is individual unit information that is distinguished from each other, such as a first examination, a second examination, first medicine, and second medicine and is defined in the system) are separate objects and may be displayed such that they can be recognized. Accordingly, the user can select only some unit information 222 in one section and move the selected unit information 222 to the area in which information corresponding to the reference date or after the reference date is displayed to input the information.

Here, one section 221 may be displayed including a part in which a plurality of pieces of unit information is displayed and a blank part 221-1 in which additional information is not displayed. In this case, the user may select the entire corresponding section by selecting the blank part 221-1 and may select only some unit information by selecting the part in which unit information 221-2 is displayed. Such a selection operation may be performed by clicking or touching a corresponding position with a mouse.

When the entire section is selected and moved in a drag-and-drop manner, the entire content of the section is input to the input area (refer to FIG. 9). Alternatively, when only some unit information in the corresponding section is selected and moved in a drag-and-drop manner, only the content of unit information required in the input area may be selectively input.

FIG. 10 illustrates an example of selecting and inputting the second unit information 221-2 and selecting and inputting the fourth unit information 222-2. In this case, when unit information input to the prescription field corresponds to examination prescription, a new section 221z with respect to details of an examination prescribed in the examination field on the corresponding date (the reference date in the case of FIG. 10) is automatically generated as described above. In this case, the generated new section may be displayed including information on whether the examination is completed. In a state in which the examination is not in progress, the examination result may be displayed in the corresponding section as “waiting”, and in the state in which the examination is completed, the examination result may be displayed.

In the above-described embodiment, it is possible to input medical information by using a separate input window or by dragging and dropping all or some of sections. According to this input operation, a section displaying input content is displayed in the area in which information corresponding to the reference date or after the reference date is displayed. In this case, the section displaying the input content may include a plurality of pieces of unit information according to the input content. At this time, the plurality of pieces of unit information may be displayed in the input order, but the plurality of pieces of unit information may be automatically sorted and displayed according to a preset sorting criterion and may be automatically sorted and displayed whenever input operation is performed. In this case, the preset criterion may be set in various ways. For example, unit information corresponding to medicine prescription may be disposed on the upper side of the prescription field, and unit information corresponding to examination prescription may be disposed on the lower side. In this way, when input unit information is automatically aligned, it is possible to easily ascertain information input by the user through the interface.

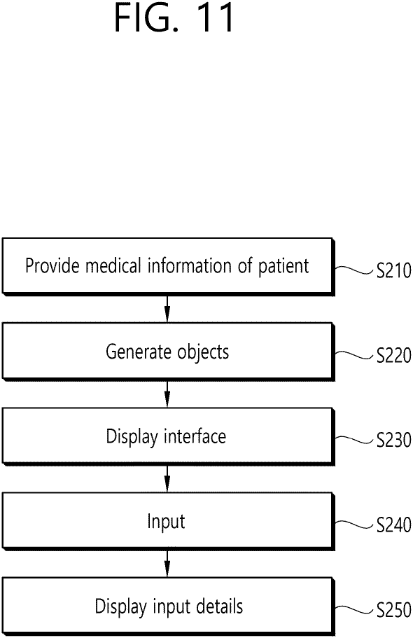

FIG. 11 is a flowchart illustrating the input operation described with reference to FIGS. 8 to 10. However, detailed description of steps similar to those of FIG. 6 described above is omitted in order to avoid redundant description, or the steps will be briefly described by being integrated or omitted.

As illustrated in FIG. 11, the data transfer unit of the processor performs a step of retrieving medical information on a patient from the database (S210). The retrieved medical information on the patient is stored in the cache storage unit, and the object generation unit generates an object for each section to be displayed on the interface using the stored medical information (S220). Then, the medical information is displayed on the interface using the generated object (S230).

The user checks the medical information on the patient through the interface and performs a step of inputting necessary information (S240). In this case, the input information may be information on medical treatment and examination performed on the reference date, or details of prescription, or may be reservation information regarding medical treatment and examination performed after the reference date. In this input step, as described above, the user can directly input the information through a separate input window or select all or some of displayed sections and move the same to the area in which information corresponding to the reference date or after the reference date is displayed in a drag-and-drop manner.

Thereafter, a step of displaying content that the user wants to input is performed (S250). In this case, when the input operation of the user is performed, the input processing unit checks information the user wants to input, and the object generation unit generates an object corresponding to the input information. In addition, the generated object is displayed in the form of a section on the interface such that the user can check the input content.

### Selective Query of Information Related to Selected Diagnosis Name

The interface according to the present embodiment has a function of extracting only medical information related to a representative value desired by a user from among various types of medical information on a patient and displaying the same.

When a patient visits a medical institution due to various lesions, medical information on the patient displayed on the interface may include diagnosis, examination, and prescription records with respect to various lesions. Therefore, when a user wants to inquire information related to a specific diagnosis name, there is a problem of having to select information because information with low relevancy is mixed, and the efficiency of inquiry may be reduced because the amount of information displayed on one screen is limited. Accordingly, in the present embodiment, when the user selects a diagnosis name as a representative value, only medical information related to the diagnosis name can be extracted and displayed, and thus user inquiry convenience and inquiry efficiency can be improved.

To this end, the interface includes a function by which a user can select a representative value. As an example, the user may select a representative value by selecting a section 221h of a diagnosis name field in which a diagnosis name of interest is displayed. Alternatively, a representative value may be selected by inputting/selecting a diagnosis name of interest through a separate selection window. Although a diagnosis name will be described as a representative value hereinafter, the present invention is not limited thereto, and it is also possible to select other items such as a medicine prescription item, a treatment prescription item, and an examination prescription item as representative values.

The processor 210 stores data serving as a criterion for determining whether or not there is relevance with each representative value selectable by the user. Accordingly, when the user selects a representative value, related information is extracted from medical information on the patient stored in the cache storage unit 212 according to the relevance determination criterion. Accordingly, the object generation unit 213 may generate an object only for the extracted information, excluding information with low relevance, and display the generated object in a section on the interface 220.

In this case, the information extracted by the processor 210 may be information corresponding to a specific field or a specific subfield. For example, if a diagnosis name with respect to a retinal disease is selected as a representative value, the processor may extract a relevant intraocular pressure field, fundus OCT examination field, blood examination field, and the like from the medical information on the patient and exclude an irrelevant lung function examination field and ultrasound examination field.

Furthermore, the processor 210 may not only extract a specific field or a specific subfield, but also extract highly relevant detailed information from information included in an extracted field. In addition, the object generation unit 213 may generate an object to display highly relevant detailed information on the interface. In this case, the object generation unit 213 may generate an object through a method defined differently from the default interface screen display method. For example, when a diagnosis name with respect to diabetes is selected as a representative value, a blood test field is extracted as a relevant field and an object may be generated such that a blood sugar level and a lipid level among blood test information are displayed in a section. Due to this, it is possible for the interface to display a problem oriented view of the user.

According to such a selective display method in consideration of the user’s interest, date information displayed on the interface may be changed differently compared with information displayed on the interface before the user selects a representative value. This is because information on dates on which medical treatment for diseases other than the representative value was performed can be excluded because the information is not relevant information. Further, among displayed date information, only relevant fields are selectively displayed. Furthermore, even if pieces of information correspond to the same field, specific relevant information may be displayed differently.

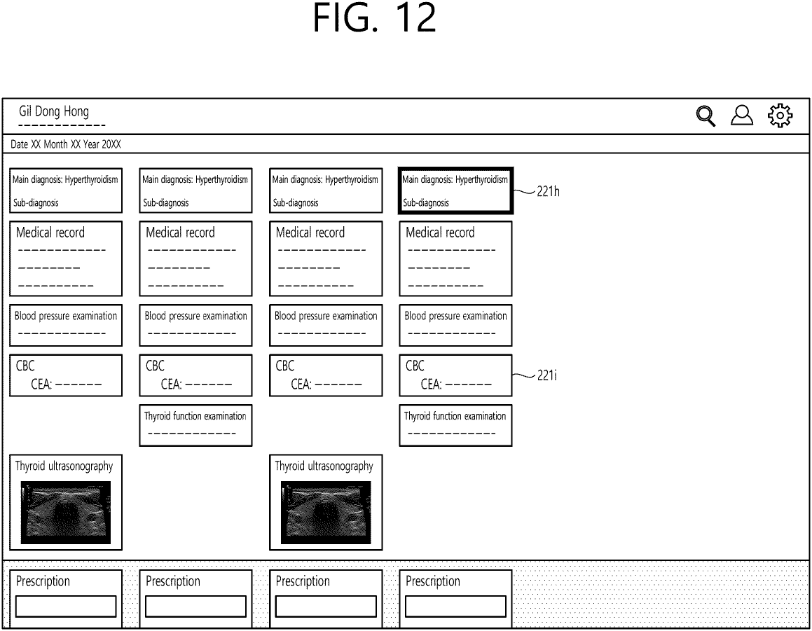

FIG. 12 is a diagram showing display content of the interface when a predetermined representative value is selected. FIG. 12 shows the interface when the user selects a diagnosis name with respect to hyperthyroidism as a representative value.

Among medical information of the corresponding patient, only medical information corresponding to dates on which the patient was diagnosed with hyperthyroidism and medical treatment was performed is extracted and displayed on the interface. However, information corresponding to a date including examination information highly related to the representative value can be displayed on the interface even if the diagnosis name of the corresponding date is different.

In addition, fields highly related to hyperthyroidism among the date information are mainly displayed on the interface. However, the diagnosis name field and the medical record field among the corresponding date information may be displayed on the interface regardless of the degree of relevance to the representative value. Specifically, as shown in FIG. 12, results of a blood pressure test, blood test, thyroid function test, and thyroid ultrasound test in the examination field, which are highly related to hyperthyroidism, are displayed on the interface. Among them, the blood test result may be displayed focusing on CEA levels highly relevant to hyperthyroidism, unlike the method shown in FIGS. 3 to 5 (refer to 221i).

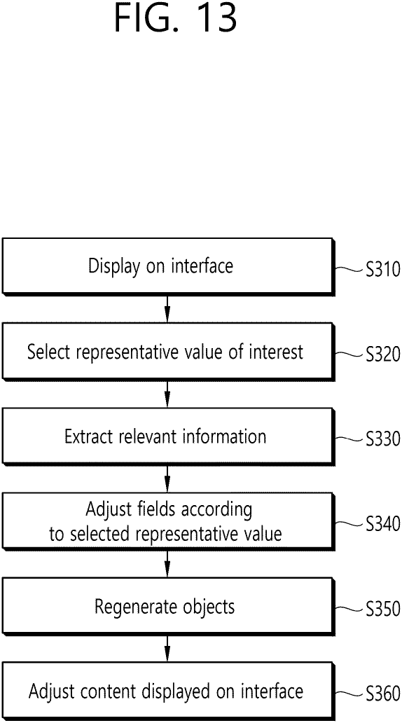

FIG. 13 is a flowchart showing a method of selectively displaying information related to a representative value selected by a user. Hereinafter, the method of displaying information related to a representative value will be described in more detail with reference to FIG. 13.

First, a step in which medical information on a patient requested by the user is retrieved from the database and displayed on the interface is performed (S310). This step has been described in detail in steps S110 to S140 of FIG. 6.

The user performs a step of selecting a representative value of interest through the interface (S320). In this case, the representative value may be a patient’s diagnosis name, and in addition to this, may be various items of interest, such as a prescribed specific medicine name, a prescribed treatment name, and the like.

When the user sets the representative value through the interface, the processor performs a step of extracting information related to the representative value from the medical information on the patient (S330). As described above, the processor 210 stores a criterion for determining relevance for each representative value, and the processor 210 extracts relevant information from the medical information on the patient stored in the cache storage unit 212 based on the criteria.

Further, a step of adjusting fields displayed on the interface may be performed (S340). In this step, displayed field items and the order thereof may be determined according to the presence or absence of information on the corresponding fields among medical information extracted in the previous step. However, since field arrangement displayed on the interface according to a selected representative value is predefined regardless of extracted medical information, fields may also be adjusted as defined. In addition, time axis display dates may also be adjusted such that information by dates including irrelevant information is excluded and information by dates including relevant information is displayed. In addition, the processor may perform processing to configure each section in the interface according to the adjusted fields and date information. Here, each section displayed on the interface may be generated according to the adjusted fields and date information at a position where information corresponding to the corresponding date and the corresponding field among the extracted information is present. However, this step is not necessarily performed as a separate step, and date information and field information may be automatically adjusted as a result of performing other steps.

The object generation unit 213 regenerates objects for displaying the extracted information in each section of the interface (S350). In this case, an object generation method may be defined differently according to the selected representative value. For example, even if an object for the same field is generated, the object may be regenerated differently through a method of directly displaying detailed information highly relevant to the representative value among information on the corresponding field to the user (for example, a highly relevant value in blood test results is displayed).

The regenerated object is displayed in each section of the interface, and thus the content displayed on the interface can be adjusted (S360). Through this step, information related to the representative value selected by the user can be divided into sections and displayed in time series on the interface.

As described above, according to the present embodiment, it is possible for the user to selectively display and inquire only information related to a representative value of interest, such as a specific diagnosis name, on the interface.

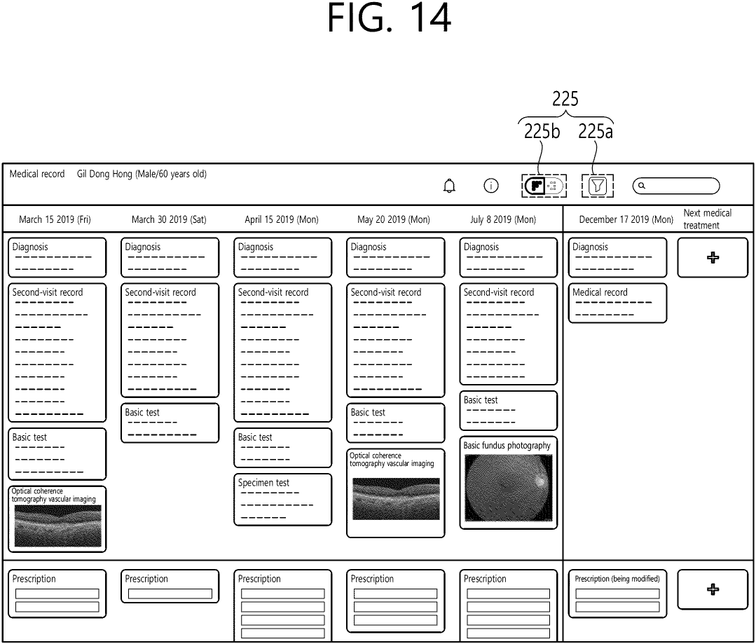

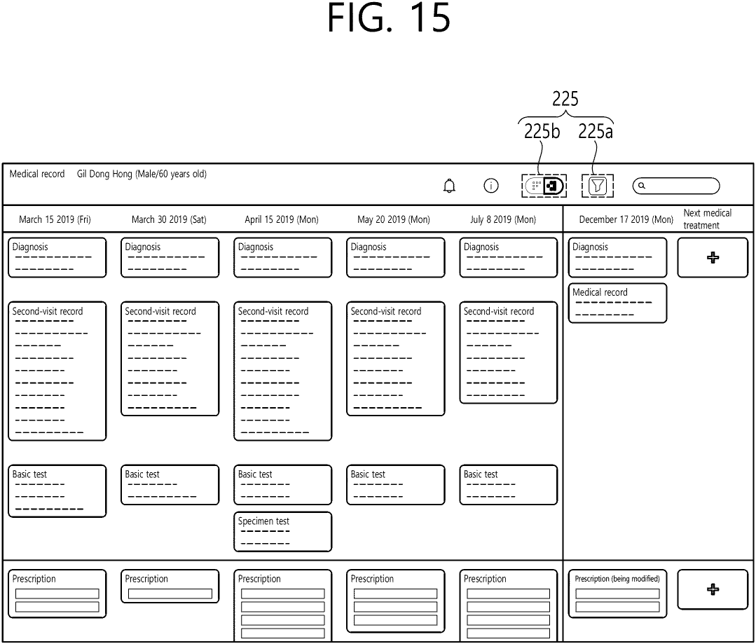

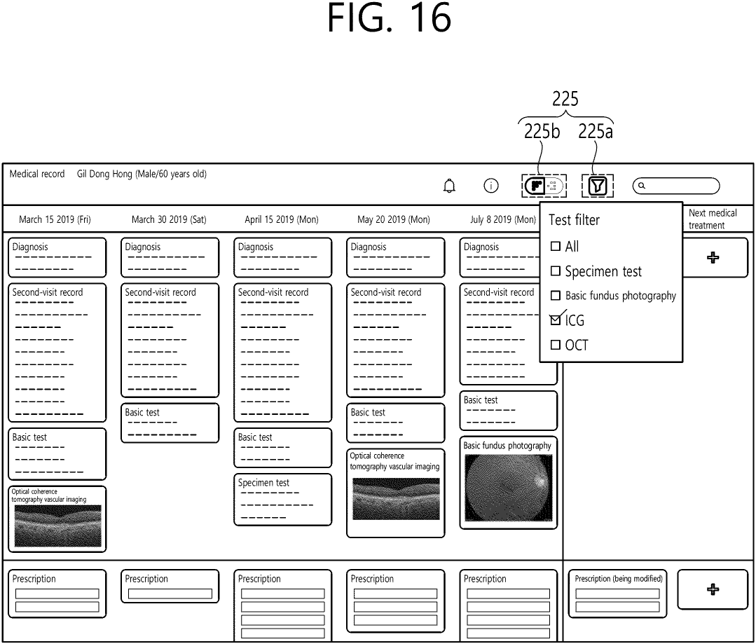

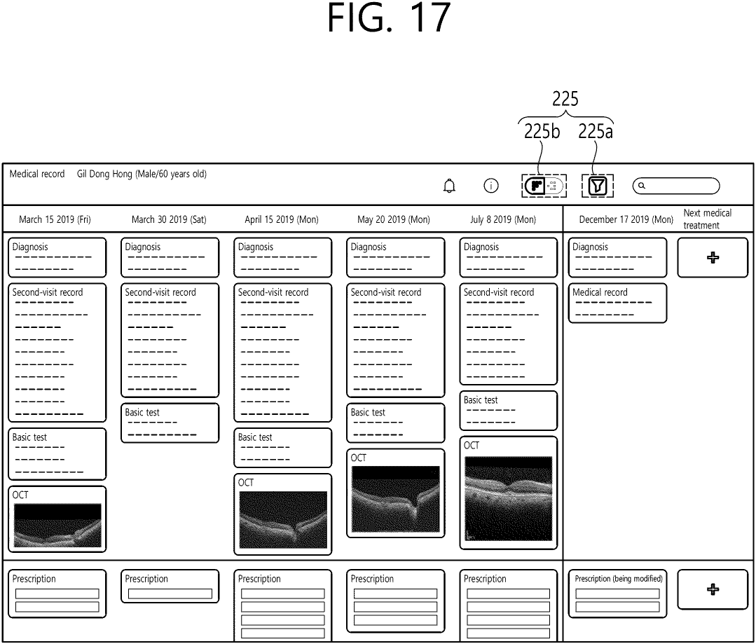

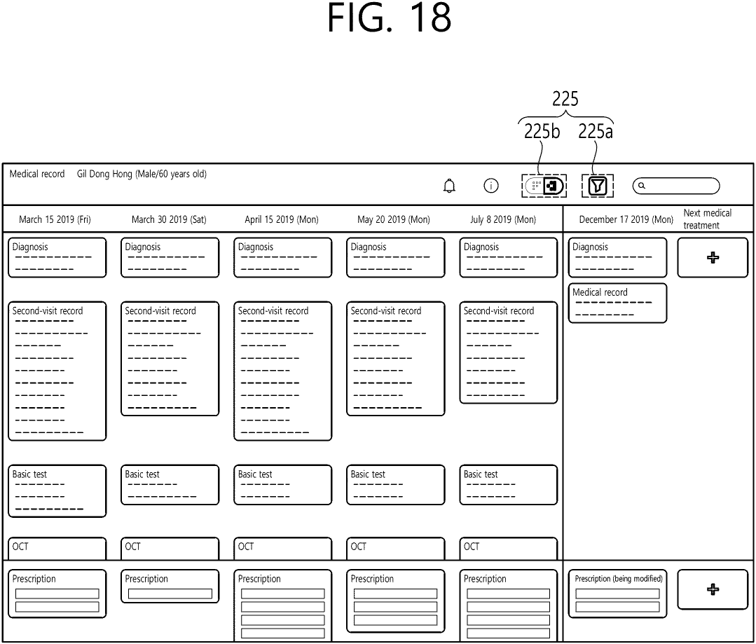

Hereinafter, various display modes of the interface according to the present embodiment will be described in detail with reference to FIGS. 14 to 18. FIG. 14 is a diagram showing a display mode in which information is aligned upward in the first display mode, FIG. 15 is a diagram showing a mode in which information is aligned by fields in the first display mode, FIG. 16 is a diagram showing setting of a filter value in the second display mode, FIG. 17 is a diagram showing a display mode in which information is aligned upwardly in the second display mode, and FIG. 18 is a diagram showing a mode in which information is aligned by fields in the second display mode.

As shown in FIGS. 14 to 18, the interface of the present embodiment includes a mode selector 225 for selecting a display mode and may display medical information in various display modes according to user selection.

In this case, the mode selector includes a first selection means 225a for selecting the first mode and the second mode, and a second selection means 225b for selecting a vertical alignment method in each mode. Here, the first selection means 225a can be selectively set to display representative value-centered selected information (information related to a representative value) among the information displayed on the interface to set the first display mode or the second display mode.

First, FIGS. 14 and 15 show examples of the first display mode. The first display mode is a default display mode in which provided medical information on a patient is displayed, and displays information of all displayable fields among the provided information on the interface. In this case, the first selection means is not selected, and when the user requests patient medical information query, the corresponding information may be displayed in the first display mode.

Specifically, in FIGS. 14 and 15, the horizontal axis corresponds to the time axis, medical information by dates is arranged in time series along the time axis, and medical information on the same date is displayed in each section according to field information in a preset field arrangement order in the vertical direction. Here, in FIG. 14, a plurality of sections corresponding to information of the same date are aligned upward and displayed, and the sections are displayed such that the vertical space between sections on the same date is minimized without a vertical axis separately defined ((1-1)-th display mode). On the other hand, in FIG. 15, the vertical axis is defined according to field items, and sections corresponding to the same field are arranged side by side rather than being aligned upward, as compared to FIG. 14 ((1-2)-th display mode corresponding to FIGS. 3 to 5).

In this case, the mode for displaying sections in upward alignment can be set by selecting the left button of the second selection means 225b, and the mode for aligning and displaying sections by fields can be set by selecting the right button of the second selection means 225b. The (1-1)-th display mode in which sections are aligned upward has an advantage in that more information can be displayed on the interface without an additional scrolling operation, whereas the (1-2)-th display mode in which sections are aligned by fields has an advantage in that information corresponding to the same field can be checked in time series and thus information change over time in the corresponding field can be easily ascertained.

FIGS. 16 to 18 show examples of the second display mode. The second display mode is a mode in which only specific information of interest of the user is selectively extracted and displayed among medical information displayed in the first display mode, and selectively displays only some of a plurality of sections displayed in the first display mode.

This second display mode may be set by selecting the first selection means 225a. As shown in FIG. 16, when the first selection means 225a is selected, a selection window is displayed, and the user can set a filter value of interest through the selection window. In this case, the filter value may be the representative value described with reference to FIGS. 12 to 13. The user may set a representative value by inputting the representative value through the selection window or by selecting at least one of representative value options displayed in the selection window.

Here, the representative value may be one of a treatment subject, a diagnosis name, an examination item, a medicine prescription item, and a treatment prescription item, as described above. When the representative value is set in this way, information corresponding to related fields may be extracted based on correlation between prestored representative values and fields and may be displayed on the interface.

In this case, field information related to a representative value may conform to prestored related information, but may be configured to be modified by the user through the selection window. Specifically, when the user inputs a representative value through the selection window, field information related to the representative value is displayed and the user can edit the fields displayed through the second mode by selecting some of the displayed related field information or adding additional field information. Furthermore, it is possible to directly select necessary field information as a filter value to determine fields displayed through the second mode without setting an additional representative value.

When the fields displayed through the second display mode are determined in this way, information of the fields may be extracted from the provided medical information on the patient to generate objects by dates and objects by fields, the time axis of the interface may be adjusted in consideration of date information omitted according thereto, and then the extracted information may be displayed in the form of a plurality of sections on the interface. In addition, the content of a section corresponding to the same field on the same date may be displayed in a different manner from that in the first display mode in consideration of relevance to the representative value. Since these steps are performed similarly to the steps described with reference to FIGS. 12 to 13, a detailed description thereof will be omitted.

FIGS. 17 and 18 show interfaces displayed in the second display mode through the above-described steps. Here, the second display mode may also be configured to align information upward and display the same or to align information by fields and display the same using the second selection means 225b. FIG. 17 shows a state in which the left button of the second selection means 225b is selected and information is aligned upward and displayed ((2-1)-th display mode), and FIG. 18 shows a state in which the right button of the second selection means 225b is selected and information is aligned by fields and displayed ((2-2)-th display mode).

As described above, the interface of the present embodiment can provide the first display mode for displaying all medical information on a patient and the second display mode for displaying some medical information of interest according to user setting and display medical information in upward alignment or alignment by fields in the display modes.

### Display of Examination Field Information

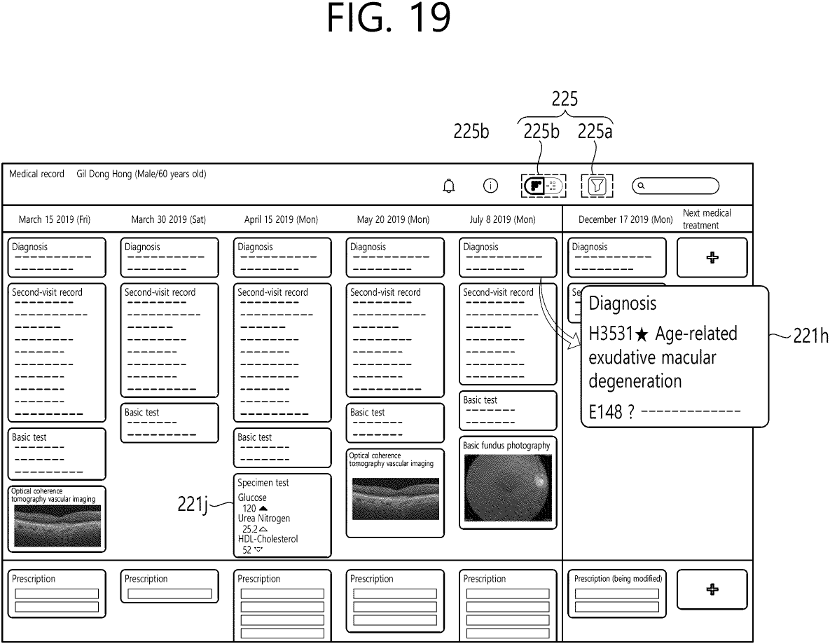

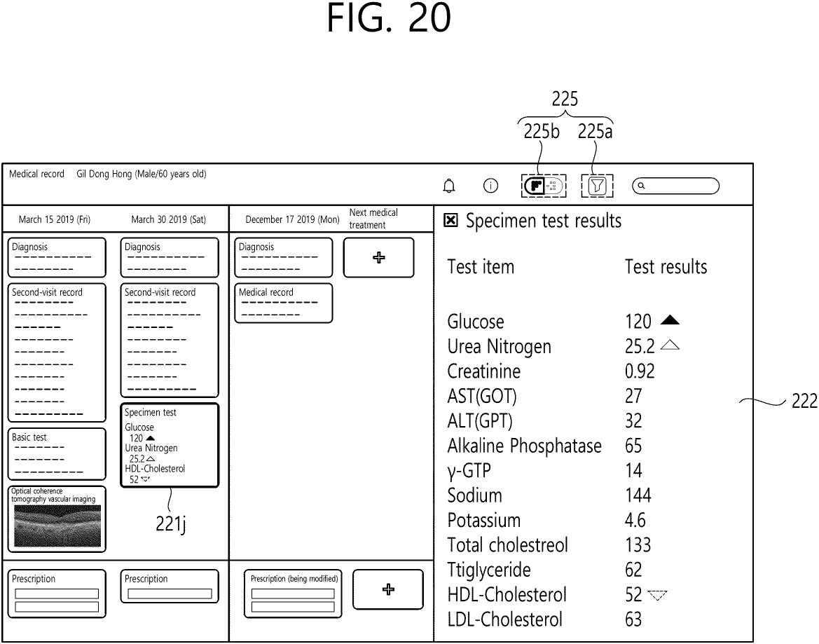

FIG. 19 is a diagram showing an example of an interface in which diagnosis and examination information is displayed, and FIG. 20 is a diagram showing an example of an interface in which examination information is displayed through a detailed inquiry window. As described above, when medical information on a patient is provided, the object generating unit 213 processes data of the information such that the information can be displayed in a manner defined according to field type at the time of processing the information in order to display the information in the form of a section on the interface.

For example, the section 221h corresponding to the diagnosis information field is configured to additionally display a sub-diagnosis name (additional diagnosis) or a similar symptom as well as a main diagnosis name (main condition). In this case, the section corresponding to the diagnosis information field displays the main diagnosis name and the sub-diagnosis name, or the main diagnosis name and similar symptom in a distinguishable manner using any one of a symbol, a color, and a letter shape. As an example, as shown in FIG. 19, the main diagnosis name is marked with an asterisk at the end of the diagnosis name and the sub-diagnosis name or similar symptom is marked with a question mark at the end of the diagnosis name such that the user can distinguish and identify them.

In addition, in the examination field, some subfields in which examination results are displayed as numerical values are configured to display not only examination results on the corresponding date but also comparison with results of the same examination performed before the corresponding date in the corresponding section.

As an example, a specimen test subfield section shown in FIGS. 19 and 20 will be mainly described. FIG. 20 illustrates a state in which detailed information corresponding to an examination information field section 221j of a specific date of FIG. 19 is displayed through the detailed query window 222. As displayed in the detailed query window 222 of FIG. 20, specimen test results include test results with respect to various items. In the section 221j displayed on the dual interface, results of main items among the various items may be extracted and displayed. For example, as shown, only three items corresponding to a glucose level, a urea nitrogen level, and an HDL-cholesterol level among 10 test items may be displayed through the corresponding section.

Here, the test results of the corresponding date are displayed in numbers on the section. Furthermore, it is possible to display whether a result has increased or decreased compared to a previous test result with an arrow. For example, an upward arrow may be displayed when the test result of a corresponding item has increased compared to a previous test result, and a downward arrow may be displayed when the test result of the corresponding item has decreased compared to the previous test result.

Furthermore, whether the corresponding test result is normal may be displayed using a color. In this case, the color may be the color of a number or the color of the arrow. For example, if the test result is normal, it may be displayed in black, and if the test result is out of a normal range, it may be displayed in a color other than black. For example, in a case of abnormality that exceed an upper limit of the normal range, the test result may be displayed in red, and in a case of abnormality that does not reach a lower limit of the normal range, the test result may be displayed in blue to indicate not only whether the test result is normal but also how much the test result deviates from the normal range. However, FIG. 20 shows an example in which test results are displayed using the pattern and shading of an arrow instead of colors for drawing display. In this case, the user can simultaneously check not only test result values but also comprehensive information about patient’s status changes and normality/abnormality using minimum display information displayed in the examination information section.

In this way, the object generation unit 213 creates an object such that the object can be displayed in a section in a manner defined for each field. For example, when the object generation unit generates an object of the above-described examination result field, the object generation unit generates the object such that examination result numerical values of the corresponding date can be reflected and whether the values have increased or decreased compared to previous (immediately previous) examination results and results of comparison with a normal range can be displayed. In this case, the object generation unit may perform an operation of checking whether the values have increased or decreased compared to the previous examination results or comparing the test result with the normal range and display the same, or examination information including the operation results may be retrieved from the database to a user terminal.

In addition, although the object generation unit generates an object to display a section in a manner defined for each field, as described above, if there is a result that deviates from the normal range among examination items, the object generation unit can generate an object to display the corresponding examination item in the section. Specifically, in generation of an object corresponding to the examination information section, the object generation unit may generate objects such that a glucose level, a urea nitrogen level, and an HDL-cholesterol level can be displayed in the section, or if an examination result corresponding to abnormality among other examination items is included, may generate objects such that the examination item including the abnormal result is displayed in the section with priority over the defined items.

Although the method of displaying information in sections using arrows and colors as well as numerical values has been mainly described above, it is also possible to display each examination item displayed in the detailed query window 222 in the above-described manner.

### Display of Examination Result Graphs

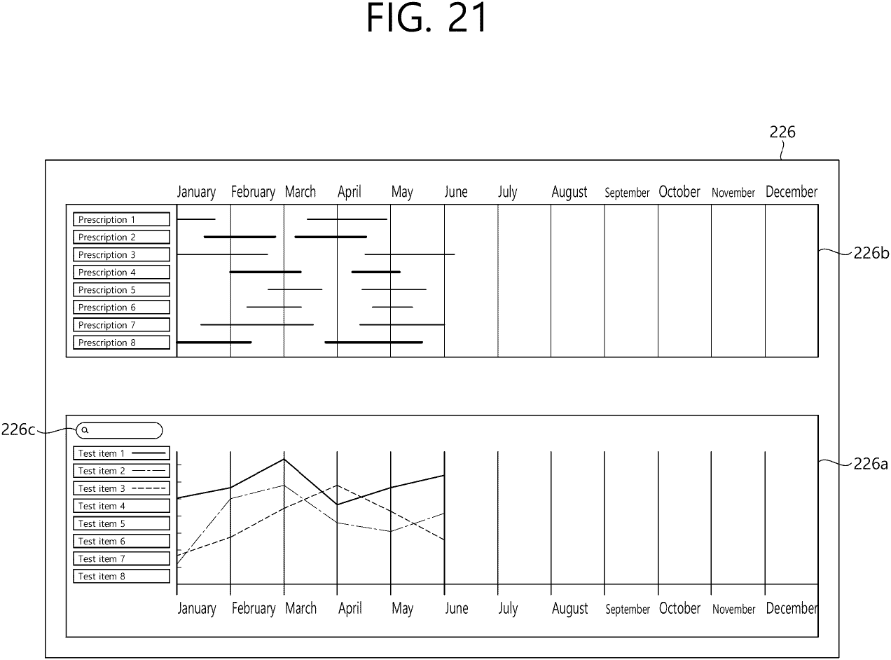

FIG. 21 shows graphs of results with respect to a specific examination item. Although it is possible to display test result changes as a single graph as shown in FIG. 5, it is also possible to configure an interface such that a correlation between test result changes and prescription information can be ascertained, as shown in FIG. 21.

Referring to the graphs shown in FIG. 21, a field name of a specific examination field may be selected and examination results of the examination field may be displayed through a separate detailed query window. Alternatively, a separate mode may be provided, the interface may be switched to a separate page for inquiring test results, an examination item may be selected through the page, and examination results of the selected item may be displayed through the interface.

Specifically, as shown in FIG. 21, the interface includes a graph display area 226a for displaying examination results and a medical record display area 226b for displaying a patient’s medical record.

The horizontal axis of the graph area 226a is a time axis, and the vertical axis is an axis indicating numerical values of examination results, and changes in examination values of a patient over time are displayed according to the horizontal and vertical axes. Here, the horizontal axis may be displayed on a monthly or weekly basis, and in addition, examination dates or an examination frequency may be displayed.

The graph display area 226a includes a selection window 226c, and the user can select an examination type to inquire through the selection window. As an example, FIG. 21 shows a result of selecting a CBC test. Some tests, such as the CBC test, include multiple detailed test items (white blood cell count, red blood cell count, red blood cell index, hemoglobin concentration, etc.), and these detailed test items are displayed on one side of the graph. The user can select a detailed test item to check and check test results for each detailed item. In this case, test results of one detailed item may be displayed in the graph area, and when the user selects a plurality of detailed items, a plurality of detailed item test results may be displayed in an overlapping manner with different colors or dot patterns. Alternatively, results of a plurality of detailed items included in the corresponding test may be displayed in an overlapping manner in the graph area regardless of whether the user selects or not.

Meanwhile, in FIG. 21, the medical record display area 226b may display a prescription record for a patient. For example, the vertical axis may indicate the type of medicine prescribed to the patient, and the horizontal axis may indicate the time axis, thereby displaying the patient’s prescription record for each period. Here, the time axis of the medical record display area is the same as the time axis of the graph area in terms of scale.

In FIG. 21, a type of prescribed medicine may be a prescription record. Therefore, in analyzing the cause of rising or falling trend of a specific test result displayed in the graph area, it is possible to simultaneously check medicine prescription records for each period. This makes it possible to check how a particular prescription affects the patient’s condition (for example, whether corresponding medication is effective for the patient) or to predict the cause of changes in the patient’s condition (test results).

Although medicine prescription details are mainly displayed in the medical record display area in FIG. 21, various medical records that may be correlated with test results, such as test prescription and surgery history, may be displayed in addition to medicine prescription.

Furthermore, when the user selects one of treatment information and prescription information, a graph of detailed examination items associated with the selected treatment information or prescription information may be highlighted and displayed such that the graph can be identified discriminately from graphs of other detailed examination items. Alternatively, when the user selects a specific detailed examination item, a prescription item associated with the selected examination item may be highlighted and displayed to be distinguished from other prescription items. Here, a highlighted display method may be a method of changing a color or a thickness of a line. Accordingly, the user can more easily ascertain a correlation between the patient’s medical record and test results and sensitivity thereof.

In order to implement the above-described embodiment, the present invention provides a medical information query and input method including: retrieving medical information including examination information on a patient before a reference date from a database; generating objects by dates and by fields from the received medical information on the patient; displaying the objects in the form of separate sections by dates on an interface of the terminal; and displaying examination result information by dates corresponding to an examination field selected by a user as a graph through a separate display window.

In the displaying examination result information as a graph, when the user requests display of a specific examination result, the processor extracts examination results and medical record information to be displayed from the medical information on the patient retrieved from the database. Here, the extracted medical record information may be information such as a medicine prescription, an examination prescription, and a surgery history, and the medical record information corresponding to a period displayed on an examination result graph is extracted. Then, results of detailed examination items and the medical record information are schematically displayed on the same time axis using examination result information and the extracted medical record information, as shown in FIG. 21.

In this case, the medical information on the patient retrieved from the database may include information on whether medical record information (for example, prescription information) is related to an examination item (for example, medicine prescription data for diabetes treatment and blood sugar test data include tags of the same group). Accordingly, when the user selects a specific prescription item or examination item displayed on the interface, the processor may control a relevant examination item or prescription item such that it is highlighted and displayed.

The medical information query and input method according to the above-described embodiment of the present invention may be implemented as a program (including an application) to be executed in association with a user terminal and stored in media.

This program may include code encoded in a program language readable by an arithmetic unit of a terminal in order for a user terminal to read the program and execute the above-described methods implemented in the program. This code may include functional code associated with functions that define functions necessary to execute the above-described methods and may include execution procedure related control code necessary for an arithmetic unit of a terminal to execute the aforementioned functions according to a predetermined procedure.

In addition, media in which the program is stored are not media storing data for a moment, such as a register, a cache, and a memory, and are media that semipermanently store data and are readable by devices. Specifically, examples of the media include a ROM, a RAM, a CD-ROM, a magnetic tape, a floppy disk, and an optical data storage device, and the media are not limited thereto. For example, the aforementioned program may be stored in various recording media on various web servers accessible by user terminals. Furthermore, the media may be distributed to computer systems connected via a network and computer-readable code may be stored therein in a distributed manner.

Although an embodiment of the present invention has been described in detail, the present invention is not limited to the above-described embodiment. Those skilled in the art will appreciate that various modifications and variations can be made in the present invention without departing from the spirit or scope of the invention described in the appended claims.

What is claimed is:

1. A medical information query and input method comprising:
retrieving, from a database, medical information on a patient before a reference date;
generating a plurality of objects by extracting or processing some of the retrieved medical information such that information by dates and by fields among the retrieved medical information is able to be displayed on an interface of a terminal in a form defined for each field; and
displaying the objects in a form of separate sections on the interface.

2. The medical information query and input method of claim 1, wherein an object of a preset field among the plurality of objects is generated such that the object includes results of comparing information corresponding to a corresponding date with information before the corresponding date, and a section of the preset field on the interface displays the results of comparing the information corresponding to the corresponding date with the information before the corresponding date.

3. The medical information query and input method of claim 2, wherein the preset field is one of fields displaying examination results of the patient, and the section of the preset field displays information on an examination result corresponding to the corresponding date and information regarding increase/decrease compared to examination results before the corresponding date.

4. The medical information query and input method of claim 3, wherein the information on the examination result corresponding to the corresponding date is indicated as numbers, and the information regarding increase/decrease is indicated as a symbol.

5. The medical information query and input method of claim 3, wherein the section of the preset field further displays information on whether the examination result corresponding to the corresponding date is normal.

6. The medical information query and input method of claim 1, further comprising, when a user selects a section of a preset field on the interface, displaying detailed information of the section through a detailed display window,
wherein the amount of information displayed through the detailed display window is greater than the amount of information displayed in the section.

7. The medical information query and input method of claim 1, wherein a section corresponding to a diagnosis information field among the sections displays information on a main diagnosis name and a sub-diagnosis name or the main diagnosis name and a similar symptom.

8. The medical information query and input method of claim 7, wherein the section displays the main diagnosis name and the sub-diagnosis name or the main diagnosis name and the similar symptom in a distinguishable manner using any one of a symbol, a color, and a letter shape.

9. A medical information query and input program comprising:
an interface for displaying medical information on a patient in a form of separate sections according to date information and field information; and
a processor for processing medical information on a patient retrieved from a database such that the medical information is able to be displayed on the interface,
wherein the processor includes an object generation unit for generating a plurality of objects corresponding to respective sections by extracting or processing some of the retrieved medical information such that each section is able to be displayed on the interface in a form defined according to field information.

10. The medical information query and input program of claim 9, wherein, in generation of an object of a preset field, the object generation unit generates the object such that object includes results of comparing information corresponding to a corresponding date with information before the corresponding date, and a section of the preset field displayed on the interface is configured to display the comparison results.

11. The medical information query and input program of claim 10, wherein the preset field is one of fields displaying examination results of the patient, and the section of the preset field displays information on an examination result corresponding to the corresponding date and information regarding increase/decrease compared to examination results before the corresponding date.

12. The medical information query and input program of claim 11, wherein the information on the examination result corresponding to the corresponding date is indicated as numbers and the comparison information is indicated as a symbol.

13. The medical information query and input program of claim 11, wherein the section of the preset field further displays information on whether the examination result corresponding to the corresponding date is within a normal range.

14. The medical information query and input program of claim 9, wherein the interface is configured to, when a user selects a section of a preset field, selectively display a detailed display window for displaying detailed information of the section,
wherein the amount of information displayed through the detailed display window is greater than the amount of information displayed in the section.

15. The medical information query and input program of claim 1, wherein a section corresponding to a diagnosis information display field among the sections displays a main diagnosis name and a sub-diagnosis name or the main diagnosis name and a similar symptom, the section displaying the main diagnosis name and the sub-diagnosis name or the main diagnosis name and the similar symptom in a distinguishable manner using any one of a symbol, a color, and a letter shape.
